(19) United States
(12) Patent Application Publication
(10) Pub. No.: US 2022/0406407 A1
(43) Pub. Date: Dec. 22, 2022

(54) Deciphering Multi-Way Interactions In The Human Genome With Use Of Hypergraphs

(71) Applicant: THE REGENTS OF THE UNIVERSITY OF MICHIGAN, Ann Arbor, MI (US)

(72) Inventors: Indika RAJAPAKSE, Ann Arbor, MI (US); Stephen LINDSLY, Ann Arbor, MI (US); Can CHEN, Ann Arbor, MI (US)

(73) Assignee: THE REGENTS OF THE UNIVERSITY OF MICHIGAN, Ann Arbor, MI (US)

(21) Appl. No.: 17/839,937

(22) Filed: Jun. 14, 2022

Related U.S. Application Data
(60) Provisional application No. 63/236,744, filed on Aug. 25, 2021.
(60) Provisional application No. 63/210,678, filed on Jun. 15, 2021.

(51) Int. Cl. G16B 20/30; G16B 25/00; G16B 45/00; C12Q 1/6869

(52) U.S. Cl. CPC G16B 20/30; G16B 25/00; G16B 45/00; C12Q 1/6869

(57) ABSTRACT

A method is presented for analyzing interactions in a human genome. The method includes: receiving a biological sample of a cell from a subject; extracting read data from the biological sample, where the read data includes a set of reads; and constructing, by a computer processor, a hypergraph from the read data, where each node in the hypergraph represents a locus and hyperedges in the hypergraph represent interactions between two or more loci. The hypergraphs may be used for different applications including determining entropy, comparing different biological samples and reporting multi-way contacts in a set of transcription clusters.

### CROSS-REFERENCE TO RELATED APPLICATIONS

This application claims the benefit of U.S. Provisional Application No. 63/236,744, filed on Aug. 25, 2021 and 63/210,678, filed on Jun. 15, 2021. The entire disclosures of each of the above applications are incorporated herein by reference.

### GOVERNMENT CLAUSE

This invention was made with government support under FA9550-18-1-0028 awarded by the Air Force Office of Scientific Research (AFOSR) and under 140D6319C0020 awarded by the U.S. Department of Defense, Defense Advanced Research Projects Agency. The government has certain rights in the invention.

### FIELD

The present disclosure relates to techniques for analyzing multi-way contacts in the human genome with the use of hypergraphs.

### BACKGROUND

Genome function and genome architecture and structural features of chromatin act as modulators of genome activity. The organization of the genome is non-random and has a high degree of order. Examples of this include euchromatic and heterochromatic regions, topologically associated domains, and positioning of genes within the nucleus. Despite this, there is a large amount of variability in genome organization, where individual cells can have different genome organizations yet have similar functional outputs.

Hi-C data can be used to observe structural features through the aggregation of pair-wise contacts genome-wide, but these features cannot be captured directly. Multi-way contacts from Pore-C data can be used to unambiguously observe higher order structural features, where instances of nearby multiple genomic loci are captured together as single reads.

The organization of the genome is non-random and has a high degree of order. The current standard for experimentally capturing the genome's organization is through genome wide chromosome conformation capture (e.g., Hi-C data). Pore-C is a recently developed sequencing technology. Pore-C data contains the information of Hi-C data, but also includes multi-way interactions which cannot be directly derived from Hi-C data. Hi-C data is often used to observe structural features through the aggregation of pairwise contacts genome-wide, but these features cannot be captured directly. Multi-way contacts from Pore-C data can be used to unambiguously observe higher order structural features, where instances of nearby multiple genomic loci are captured together as single reads. In this disclosure, Pore-C data is used in the form of hypergraphs to quantify entropy of genome structure and to compare the genomes of different cell types. In addition, Pore-C data is integrated with multiple other data modalities to find biologically important multi-way interactions. While reference is made throughout this disclosure to Pore-C data, it is readily understood that the techniques described herein are applicable to other types of read data which captures multi-way interactions, especially long read data.

The intricate folding of the genome allows for approximately two meters of DNA to fit within a cell nucleus while remaining accessible for transcription. The folding patterns of the genome, or genome structure, is a rapidly advancing study. With the advent of experimental techniques based on chromosome conformation capture (3C), we have uncovered an immense amount of knowledge about how the genome is organized and how it affects genome function. Many of the advancements in this field have focused on expanding the amount of interactions between genomic loci that can be captured. Originally, 3C could only capture an interaction between two genomic loci. This was later extended to interactions from one locus to all others (4C), many loci's interactions with many others (5C), and eventually all loci to all loci (Hi-C). While extraordinarily useful in their own right, all of these technologies are only able to capture interactions between pairs of loci.

Recent advances in sequencing technologies has brought forth the ability to capture multiple loci at once genome-wide. Pore-C reads contain fragments from multiple interacting loci at once, allowing for new methods of analysis on genome structure. One can use the multi-way contacts from Pore-C reads to construct hypergraphs. Hypergraphs are similar to graphs, but instead of each edge containing two nodes, hyperedges can contain any number of nodes. This disclosure considers genomic loci as nodes in a hypergraph, and multi-way contacts as hyperedges. Incidence matrices are used to represent hypergraphs, where rows in the incidence matrices represent genomic loci and columns contain individual hyperedges. From this representation, one is able to make quantitative measurements of the genome's organization through hypergraph entropy, compare different cell types through hypergraph distance, and identify functionally important multi-way contacts in multiple cell types.

This section provides background information related to the present disclosure which is not necessarily prior art.

### SUMMARY

This section provides a general summary of the disclosure, and is not a comprehensive disclosure of its full scope or all of its features.

In one aspect, a method is presented for analyzing interactions in a human genome. The method includes: receiving a biological sample of a cell from a subject; extracting read data from the biological sample, where the read data includes a set of reads; and constructing, by a computer processor, a hypergraph from the read data, where each node in the hypergraph represents a locus and hyperedges in the hypergraph represent interactions between two or more loci.

In one embodiment, the read data has a length in range of 100 to 500 base pairs. In other embodiments, the read data has a length selected from one of 100,000 base pairs, one million base pairs or 25 million base pairs.

In some embodiments, the method further includes constructing an incidence matrix from the read data; constructing a Laplacian matrix for the incidence matrix; computing eigenvalues of the Laplacian matrix using eigen decomposition; normalizing the eigenvalues of the Laplacian matrix; and determining entropy of the hypergraph using the normalized eigenvalues. The eigenvalues of the Laplacian matrix may be normalized such that Σλᵢ=1 and entropy is computed using Shannon entropy formula, where λᵢ is an eigenvalue of the Laplacian matrix.

In another aspect, a method is presented for analyzing interactions in a human genome. The method includes: receiving a first biological sample of a cell from a subject; extracting read data from the first biological sample, where the read data includes a set of reads; constructing a first hypergraph from the read data, where each node in the first hypergraph represents a locus and hyperedges in the first hypergraph represent interactions between two or more loci; receiving a second biological sample of a cell from the subject; extracting read data from the second biological sample, where the read data includes a set of reads; constructing a second hypergraph from the read data, where each node in the second hypergraph represents a locus and hyperedges in the second hypergraph represent interactions between two or more loci; and comparing the first hypergraph to the second hypergraph by computing a distance between the first hypergraph and the second hypergraph.

In one embodiment, the first biological sample is taken from a cell having a first cell type and the second biological sample is taken from a cell having a second cell type different from the first cell type. In other embodiments, the first biological sample is taken from a cell having a given cell type at a given time and the second biological sample is taken from a cell of the subject having the same cell type but at a time different than the given time.

In some embodiments, the method further includes: constructing a first incidence matrix for the first hypergraph; constructing a first normalized Laplacian matrix for the first incidence matrix; computing a first set eigenvalues of the first normalized Laplacian matrix using eigen decomposition; constructing a second incidence matrix for the second hypergraph; constructing a second normalized Laplacian matrix for the second incidence matrix; computing a second set of eigenvalues of the second normalized Laplacian matrix using eigendecomposition; and computing the distance between the first hypergraph and the second hypergraph using the first set of eigenvalues and the second set of eigenvalues. The first and the second normalized Laplacian matrix may be constructed according to

$${\tilde{L}}_{i} = I - {D}_{i}^{- \frac{1}{2}} {H}_{i} {E}_{i}^{- 1} {H}_{i}^{T} {D}_{i}^{- \frac{1}{2}} ∈ {ℝ}^{n \times n} .$$

In yet another aspect, a method is presented for identifying transcription clusters in a human genome. The method includes: receiving a biological sample of a cell from a subject; extracting read data from the biological sample, where the read data includes a set of reads; constructing a hypergraph from the read data, where each node in the hypergraph represents a locus and hyperedges in the hypergraph represent interactions between two or more loci; constructing an incidence matrix for the hypergraph; for each multi-way contact in the incidence matrix, add a given multi-way contact to a set of potential transcription clusters in case where each locus associated with the given multi-way contact is accessible and at least one locus associated with the given multi-way contact is a binding site and the binding site is an indicator of transcription; for each multi-way contact in the set of potential transcription clusters, add a particular multi-way contact to a set of transcription clusters in case where loci associated with the particular multi-way contact contains two or more expressed genes and have at least one common transcription factor; and reporting multi-way contacts in the set of transcription clusters.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a flowchart depicting a method for analyzing interactions in a human genome.

FIG. 2 illustrates the Pore-C experimental protocol which captures pairwise and multi-way contacts.

FIG. 3 is a hypergraph and an incidence matrix representing four sets of multi-way contacts within and between chromosomes.

FIG. 4 shows how the multi-way contacts can be decomposed into pairwise contacts.

FIG. 5A is an incidence matrix for a portion of chromosome 22.

FIG. 5B depict the hyperedges and read-level contacts of a subset of multi-way contacts from FIG. 5A.

FIG. 5C depicts a hypergraph constructed from the hyperedges of FIG. 5B.

FIG. 5D are contact frequency matrices constructed by separating all multi-way contacts within this region of chromosome 22 into their pairwise combinations.

FIG. 6 is an incidence matrix for chromosome 22.

FIG. 7 is an incidence matrix for the multi-way contacts between Chromosome 20 and Chromosome 22 in 1 Mb resolution.

FIGS. 8A and 8B show incidence matrices for ten most common multi-way contacts per chromosome for fibroclasts and B lymphocytes, respectively.

FIG. 9 is flowchart showing a method for computing hypergraph entropy.

FIG. 10 is a flowchart showing a method for comparing hypergraphs.

FIG. 11 is a flowchart showing a method for identifying transcription clusters in a human genome.

FIGS. 12A and 12B are diagrams for six example transcription clusters for fibroblasts and B lymphocytes, respectively.

FIG. 13 illustrates computational flow for implementing the techniques described in this disclosure.

Corresponding reference numerals indicate corresponding parts throughout the several views of the drawings.

### DETAILED DESCRIPTION

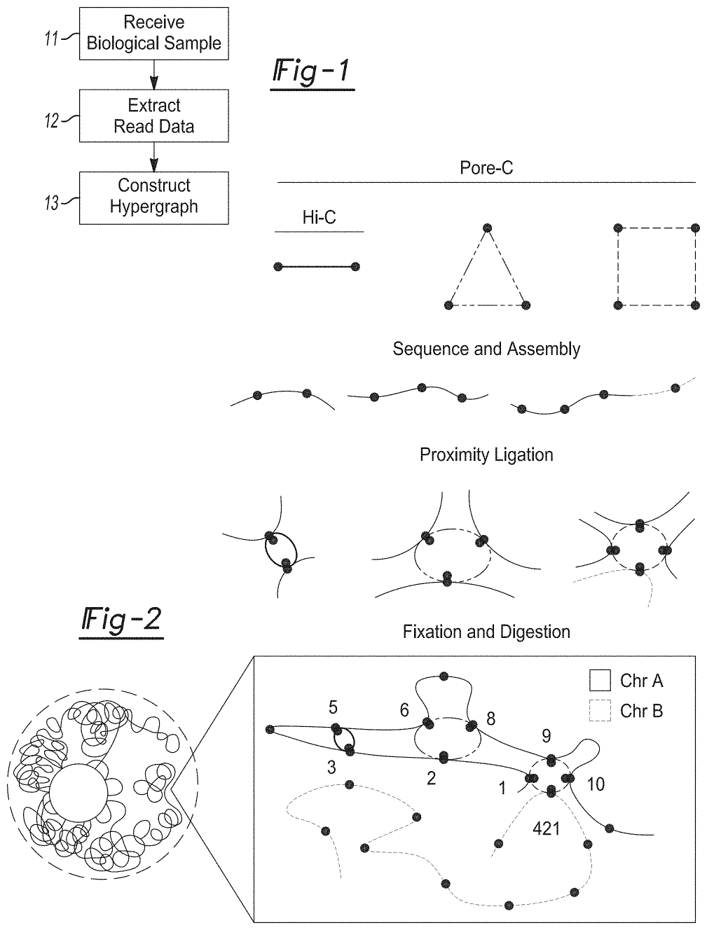

FIG. 1 depicts a method for analyzing interactions in a human genome. A biological sample of a cell from a subject is received at 11 and serves as a starting point for the analysis. Read data is extracted at 12 from the biological sample, where the read data includes a set of reads. Sequencing technologies vary in the length of the reads produced. For example, read lengths are typically in the range of 100-500 base pairs. Long read data may have lengths including but not limited to 100,000 base pairs, one million base pairs or 25 million base pairs. The broader aspects of this disclosure are not limited to read data having any particular length.

In one example, Pore-C read data was extracted from the biological sample. To obtain Pore-C read data, DNA is cross-linked to histones, digested by a restriction enzyme, ligated together, and then sequenced as shown in FIG. 2. Once these sequences are aligned to the genome, one can determine the locations where each fragment originated and construct a multi-way contact.

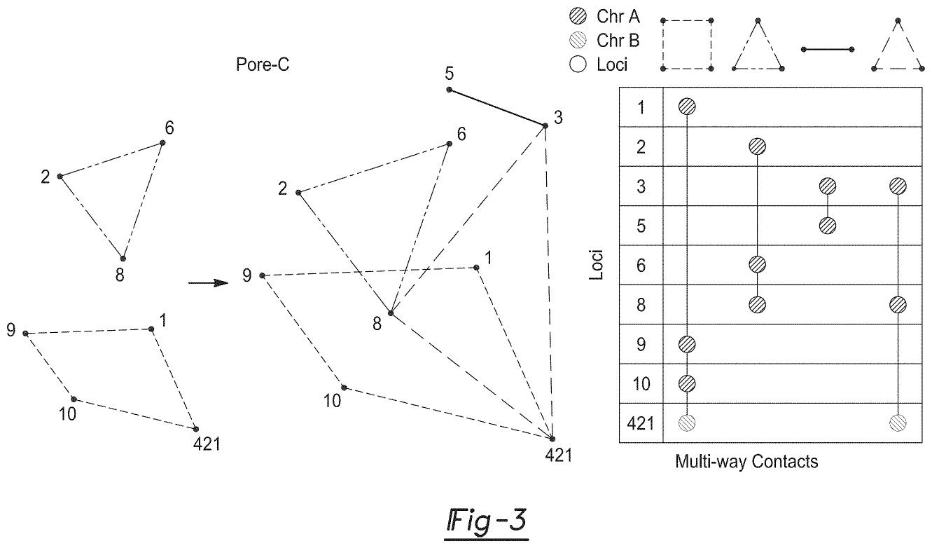

Hypergraphs are used to represent multi-way contacts as seen in FIG. 3. With reference to FIG. 1, hypergraphs are constructed at 13 from the read data, where each node in the hypergraph represents a locus and the hyperedges in the hypergraph represent interactions between two or more loci. In this way, hypergraphs provide a simple and concise way to depict multi-way contacts, and allow for abstract representations of genome structure.

Using more standard experimental techniques, such as Hi-C, adjacency matrices are often used to capture the pair-wise genomic contacts. Multi-way contacts, however, are not able to be represented in this manner, since the rows and columns of adjacency matrices only account for individual loci. In contrast, incidence matrices are used to represent multi-way contacts (FIG. 3, right). The numbers in the left column represent a bin in which a locus resides. Each vertical line represents a multi-way contact, with nodes at participating genomic loci. An example technique for constructing an incidence matrix is set forth below in Algorithm 1

Algorithm 1: Hypergraph incidence construction

| | |
|---|---|
| 1: | Input: Aligned Pore-C data |
| 2: | for each multi-way contact j do |
| 3: | if multi-way contact contains locus i then |
| 4: | H(i, j) = 1 |
| 5: | else |
| 6: | H(i, j) = 0 |
| 7: | end if |
| 8: | end for |
| 9: | Return: Hypergraph incidence matrix H ϵ [symbol] ^{n×n} where n is the |
| | total number of loci, and m is the total number of |
| | multi-way contacts. |

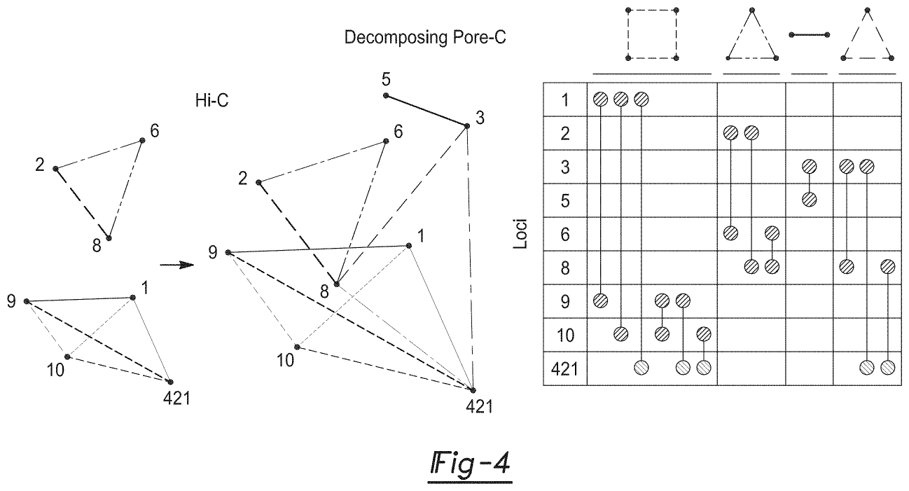

In this way, incidence matrices allow one to include more than two loci per contact and provide a clear visualization of multi-way contacts. Multi-way contacts are be decomposed into pair-wise contacts by extracting all combinations of loci as seen in FIG. 4.

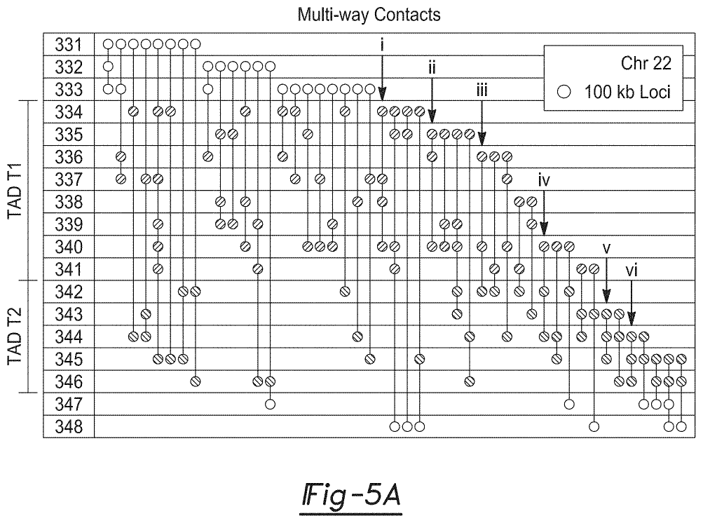

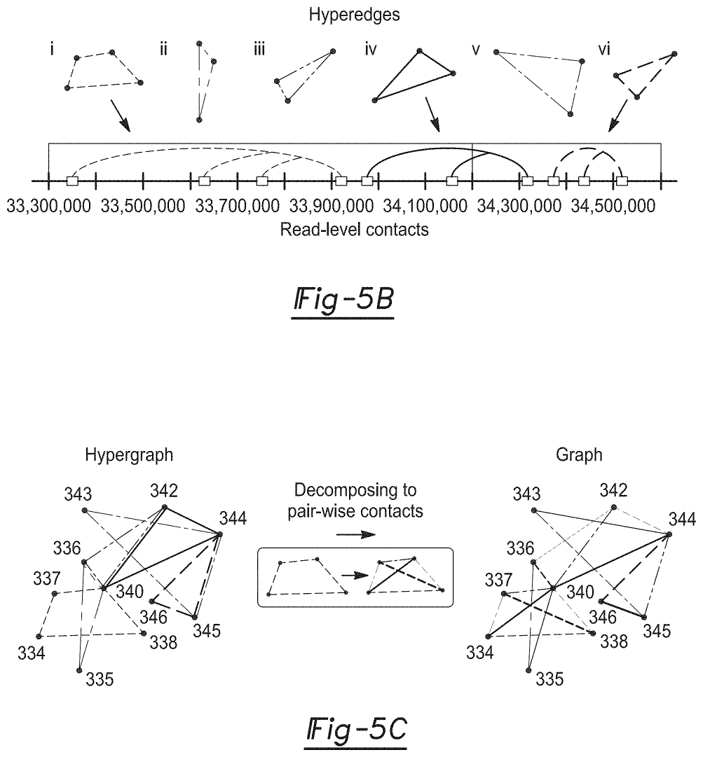

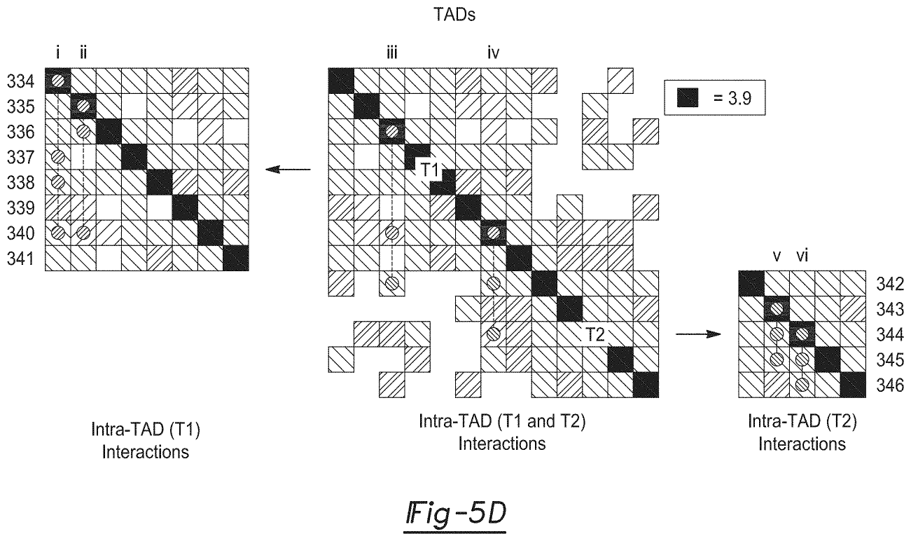

With reference to FIGS. 5A-5D, experiments were performed using adult dermal fibroblasts to demonstrate these techniques for analyzing the human genome. Input data for the experiments is publicly available Pore-C data from B lymphocytes. From this data, hypergraphs were constructed at multiple resolutions (read level, 100 kb, 1 Mb, and 25 Mb). Individual chromosomes were analyzed at 100 kb resolution and then the multi-way contacts were decomposed into their pair-wise counterparts to identify topologically associated domains.

Incidence matrix visualization of a region in Chromosome 22 from fibroblasts (V1-V4) is shown in FIG. 5A. The numbers in the left column represent genomic loci in 100 kb resolution, vertical lines represent multi-way contacts, where nodes indicate the corresponding locus' participation in this contact. The blue and yellow regions represent two TADs: T1 and T2. Six contacts, denoted by the labels i-vi, are used as examples to show intra- and inter-TAD contacts. Hyperedges and read-level visualizations of the multi-way contacts i-vi are shown in FIG. 5B, where blue and yellow rectangles (bottom) indicate which TAD each loci corresponds to. In FIG. 5C, a hypergraph is constructed using the hyperedges from FIG. 5B. The hypergraph is decomposed into its pair-wise contacts in order to be represented as a graph. In FIG. 5D, contact frequency matrices were constructed by separating all multi-way contacts within this region of Chromosome 22 into their pairwise combinations. TADs were computed from the pair-wise contacts. Example multi-way contacts i-vi are superimposed onto the contact frequency matrices. Multi-way contacts in this figure were determined in 100 kb resolution after noise reduction, originally derived from read-level multi-way contacts.

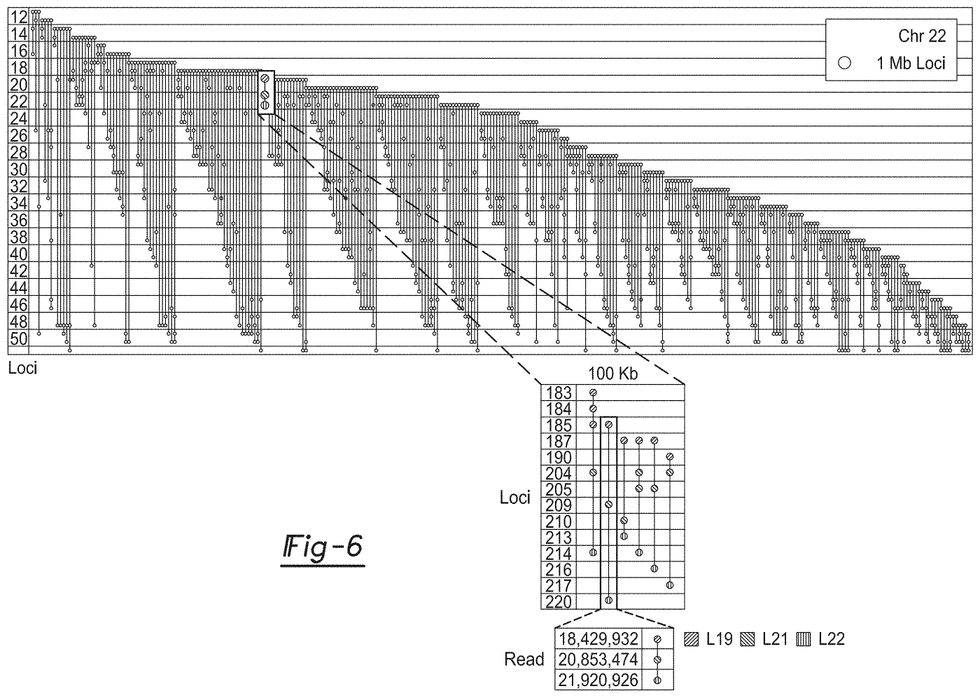

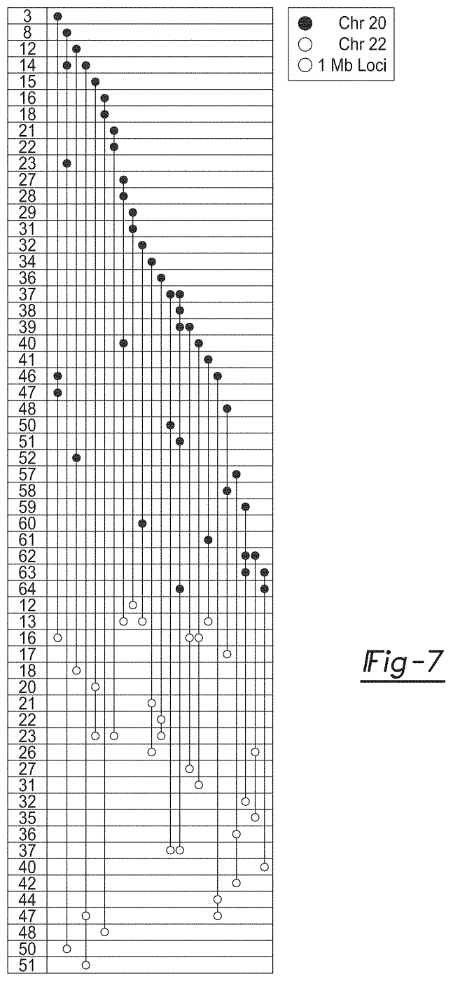

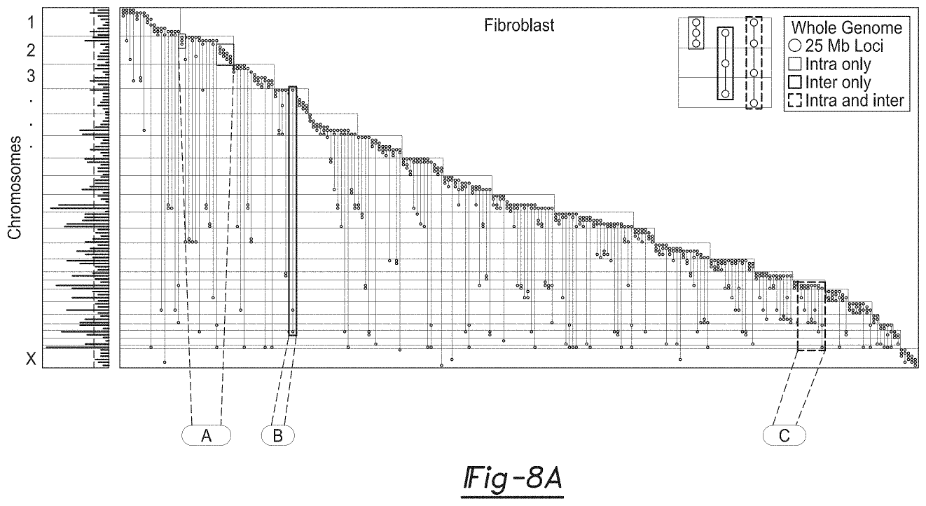

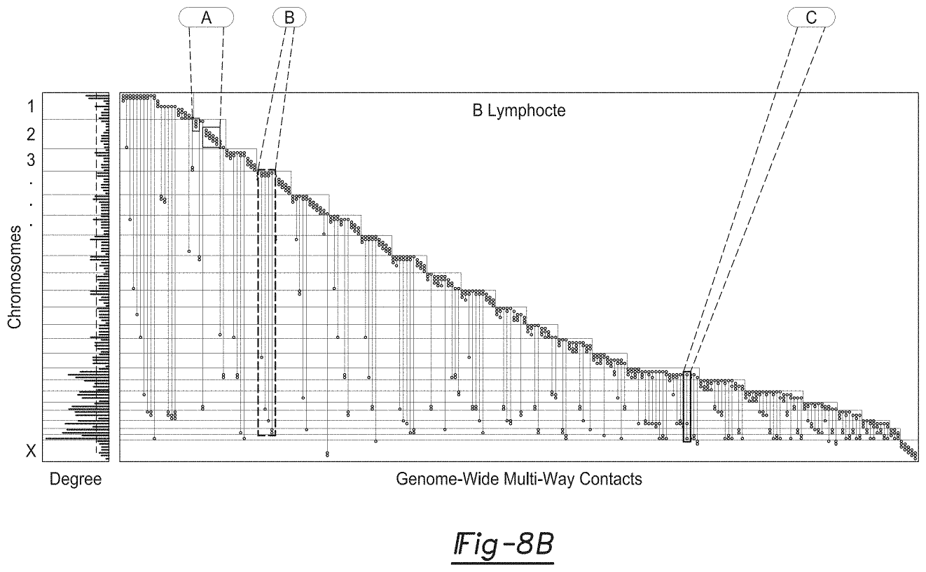

To gain a better understanding of genome structure with multi-way contacts, hypergraphs were constructed for entire chromosomes in 1 Mb resolution. The incidence matrix of Chromosome 22 is shown as an example in FIG. 6. The numbers in the left column represent genomic loci in 1 Mb resolution. Each vertical line represents a multiway contact, in which the nodes indicate the corresponding locus' participation in this contact. The zoomed in portion of the figure shows a 3-way contact to highlight how a low resolution multi-way contact can contain many contacts at higher resolutions. Specifically, the visualization shows the multi-way contact between three 1 Mb loci L19, L21 and L22 in 100 kb resolution. Multi-way contacts that contain loci from multiple chromosomes were also identified. These inter-chromosomal multi-way contacts can be seen in 1 Mb resolution in FIG. 7 as well as in 25 Mb resolution in FIGS. 8A and 8B for both fibroblasts and B lymphocytes, respectively.

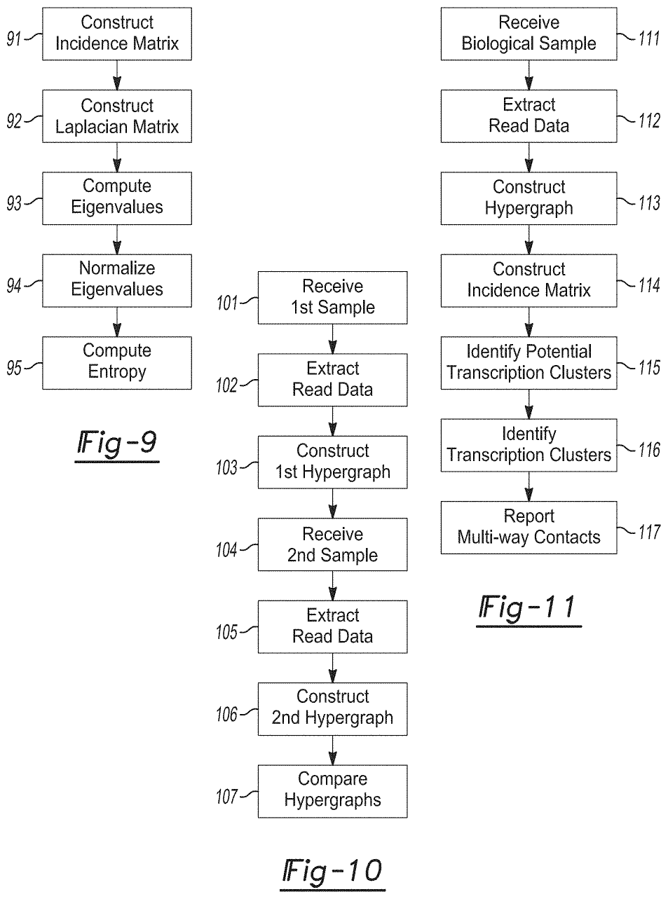

Network entropy is often used to measure the connectivity and regularity of a network. Hypergraph entropy is used to quantify the organization of chromatin structure from the read data (e.g., Pore-C data), where higher entropy corresponds to less organized folding patterns. Although there are different definitions for hypergraph entropy, one example analysis technique is further described in relation to FIG. 9.

In mathematics, eigenvalues can quantitatively represent different features of a matrix. In this example, eigenvalues of a Laplacian matrix are exploited and then fit into the Shannon entropy. That is, an incidence matrix is constructed at 91 from the read data, for example in the manner described above, and a Laplacian matrix is then constructed for the incidence matrix as indicated at 92. The incidence matrix of the genomic hypergraph is denoted by by H and the Laplacian matrix is an n-by-n matrix (n is the total number of genomic loci in the hypergraph), which can be computed by L=HH^{T} ∈R^{n×n}, where T denotes matrix transpose. Eigenvalues of the Laplacian matrix are computed at 93, for example using eigendecomposition. In some embodiments, the eigenvalues are normalized such that Σᵢ₌₁ⁿλᵢ as indicated at 94. Finally, the entropy of the hypergraph is computed at 95 using the normalized eigenvalues. More specifically, the hypergraph entropy is defined by

$$Hypergraph Entropy , = - {\sum }_{i = 1}^{n} {λ}_{i} ln {λ}_{i} \qquad ( 1 )$$

where λᵢ are the normalized eigenvalues of L, such that Σᵢ₌₁ⁿλᵢ=1, and the convention 0ln0=0 is used. Biologically, genomic regions with high entropy are likely associated with high proportions of euchromatin, as euchromatin is more structurally permissive than heterochromatin. Further details for this example definition of hypergraph entropy are set forth by C. Chen and I. Rajapakse in “Tensor entropy for uniform hypergraphs” IEEE Transactions on Network Science and Engineering, 7(4):2889-2900, 2020, which is incorporated in its entirety herein by reference. Other definitions for hypergraph entropy also fall within the broader aspects of this disclosure.

Comparing graphs is a ubiquitous task in data analysis and machine learning. There is a rich body of literature for computing graph distance with examples, such as Hamming distance, Jaccard distance, and other spectral based distances. This disclosure proposes a spectral-based hypergraph distance measure which can be used to quantify global difference between two genomic hypergraphs G₁ and G₂.

FIG. 10 illustrates the proposed technique for comparing hypergraphs. As a starting point, a first biological sample of a cell from a subject is received at 101 and a second biological sample of a cell from a subject is received at 104. In one example, the biological samples are cells of different types from the same subject. That is, the first biological sample is taken from a cell having a first cell type and the second biological sample is taken from a cell having a second cell type different from the first cell type. In another example, the biological samples are from cells of different types but from different subjects. In yet another example, the biological samples are from cells having the same type but taken from different subjects and/or under different conditions, such as at different times. One can envision receiving biological samples from other types of scenarios as well.

For each biological sample, read data is extracted from the biological sample as indicate at 102 and 105, and a hypergraph is constructed from the read data as indicated at 103 and 106. The two hypergraphs can then be compared at 107, for example by computing a distance between the two hypergraphs. Other techniques for comparing hypergraphs are also contemplated by this disclosure.

In an example embodiment, a spectral-based distance measure is used to compare the two hypergraphs. For each hypergraph, an incidence matrix is constructed and then a normalize Laplacian matrix is constructed. Denote the incidence matrices of two genomic hypergraphs by Hi E R^{nxm}^{₁} and H₂ ∈R^{nxm}^{₂}, respectively. For i=1,2, construct the normalized Lapalacian matrices as follows:

$${\tilde{L}}_{i} = I - {D}_{i}^{- \frac{1}{2}} {H}_{i} {E}_{i}^{- 1} {H}_{i}^{T} {D}_{i}^{- \frac{1}{2}} ∈ {ℝ}^{n \times n} \qquad ( 2 )$$

where I∈R^{n×n} is the identity matrix, Eᵢ∈R^{m}^{ᵢ}^{×m}^{ᵢ} is a diagonal matrix containing the orders of hyperedges along its diagonal, and Dᵢ∈R^{n×n} is a diagonal matrix containing the degrees of nodes along its diagonal. The degree of a node is equal to the number of hyperedges that contain that node. Therefore, the hypergraph distance, d, between G₁ and G₂ is defined by

$$( {G}_{1} , {G}_{2} ) = \frac{1}{n} {( {\sum }_{i = 1}^{n} {\vert {λ}_{1 j} - {λ}_{2 j} \vert}^{P} )}^{\frac{1}{P}} \qquad ( 3 )$$

where λᵢⱼ is the jth eigenvalue of \tilde{L}ᵢ for i=1,2, and p≥1. In the example embodiment, p=2 although other values may be considered. In this way, the hypergraph distance is used to compare two genomic hypergraphs in a global scale since the eigenvalues of the normalized Laplacian are able to capture global connectivity patterns within the hypergraph.

Genes are transcribed in short sporadic bursts and transcription occurs in localized areas with high concentrations of transcriptional machinery. This includes transcriptionally engaged polymerase and the accumulation of necessary proteins, called transcription factors. Multiple genomic loci can colocalize at these areas for more efficient transcription. In fact, it has been shown using fluorescence in situ hybridization (FISH) that genes frequently colocalize during transcription. Simulations have also provided evidence that genomic loci, which are bound by common transcription factors, can self-assemble into clusters, forming structural patterns commonly observed in Hi-C data. These instances of highly concentrated areas of transcription machinery and genomic loci are referred to herein as transcription clusters. The colocalization of multiple genomic loci in transcription clusters naturally leads to multi-way contacts, but these interactions cannot be fully captured from the pair-wise contacts of Hi-C. Multi-way contacts derived from Pore-C and similar read data can detect interactions between many genomic loci, and are well suited for identifying potential transcription clusters.

FIG. 11 further depicts a technique for identifying transcription clusters in a human genome. Upon receiving a biological sample of a cell from a subject at 111, an incidence matrix is constructed at 114. To construct the incidence matrix, read data is extracted from the biological sample at 112 and a hypergraph is constructed from the read data at 113 in the manner described above.

From the incidence matrix, potential transcription clusters are identified at 115. More specifically, each locus in the incidence matrix (i.e., multi-way contact) is queried for chromatin accessibility and binding. For a given multi-way contact, the given multi-way contact is added to a set of potential transcription clusters in the case where each locus associated with the given multi-way contact is accessible and at least one locus associated with the given multi-way contact is a binding site. Locus accessibility can be determined from chromatin accessibility data. In the example embodiment, the chromatin accessibility data is derived from the biological sample using Assay for Transposase-Accessible Chromatin sequencing (ATAC-seq). Chromatin accessibility data can be derived using other techniques including but not limited to DNase-seq and MNase-seq. Determining whether a given locus is a binding site can be determined from binding data, such as RNA Pol II data. In the example embodiment, the binding data is derived from the biological sample using ChIP-seq although other techniques are contemplated as well. Although not limited thereto, chromatin accessibility and transcription factor binding sites are preferably queried ±5 from the gene's transcription start site.

From the set of potential transcription clusters, transcription clusters are identified at 116. To do so, each multi-way contact in the set of potential transcription clusters is further evaluated. That is, each multi-way contact is queried for nearby expressed genes. For a given multi-way contact, the given multi-way contact is added to a set of transcription clusters in the case where loci associated with the particular multi-way contact contains two or more expressed genes and have at least one common transcription factor. Loci that contains two or more expressed genes is determined from gene expression data, for example obtained using RNA-seq or similar methods.

In the example embodiment, genes having common transcription factors were determined through binding motifs. For demonstration purposes, transcription factor binding site motifs were obtained from “The Human Transcription Factors” data. FIMO (https://meme-suite.org/meme/tools/fimo) was used to scan for motifs within ±5 kb of the transcription start sites for protein-coding and microRNA genes. The results were converted to a 22,083×1,007 MATLAB table, where rows are genes, columns are transcription factors, and entries are the number of binding sites for a particular transcription factor and gene. The table was then filtered to only include entries with three or more binding sites in downstream computations. This threshold was determined empirically and can be adjusted by changes to the provided MATLAB code. This method for identifying transcription clusters is also set forth below

Algorithm 4: Identification of Transcription Clusters

| | |
|---|---|
| 1: | Input: Hypergraph incidence matrix H, gene expression R |
| | (RNA-seq), RNA Pol II P (ChIP-seq), chromatin accessibility C |
| | (ATAC-seq), transcription factor binding motifs B |
| 2: | for each multi-way contact J in H do |
| 3: | if all loci are accessible from C and 1 locus has Pol II binding |
| | from P then |
| 4: | mult-way contact j from H is added to the set of potential |
| | transcription clusters Tₚ |
| 5: | end if |
| 6: | end for |
| 7: | for each potential transcription cluster k in Tₚ do |
| 8: | if loci contain 2 expressed genes from R which have 1 |
| | common TFs from B then |
| 9: | multi-way contact k from Tₚ is added to the set of |
| | transcription clusters T_{c} |
| 10: | end if |
| 11: | if loci contain 2 expressed genes from R which have 1 |
| | common MRs from B then |
| 12: | multi-way contact k from Tₚ is added to the set of |
| | transcription clusters Tₛ |
| 13: | end if |
| 14: | end for |
| 15: | Return: Potential transcription clusters Tₚ, transcription clusters |
| | T_{c}, and specialized transcription clusters Tₛ |

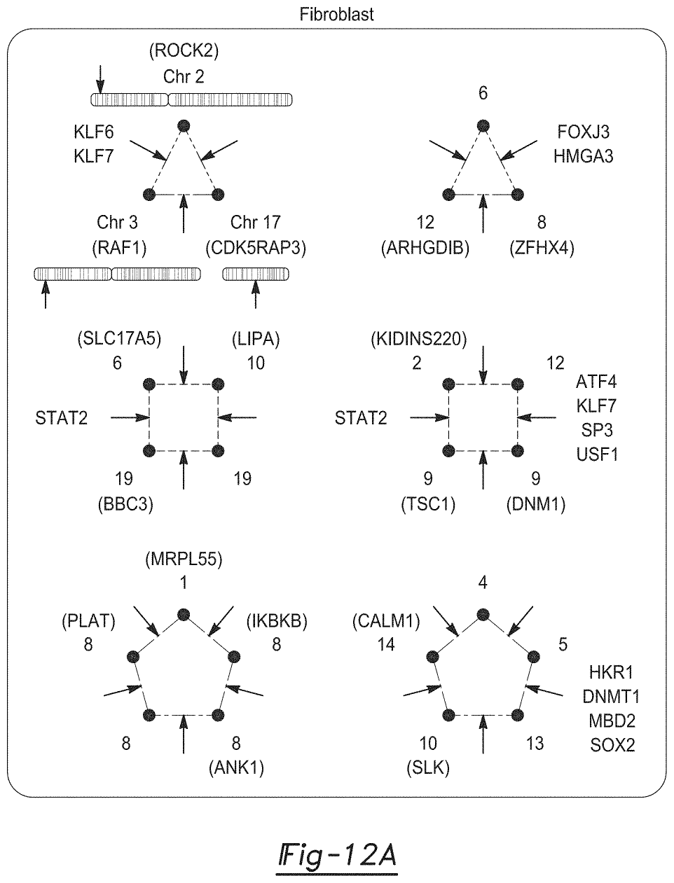

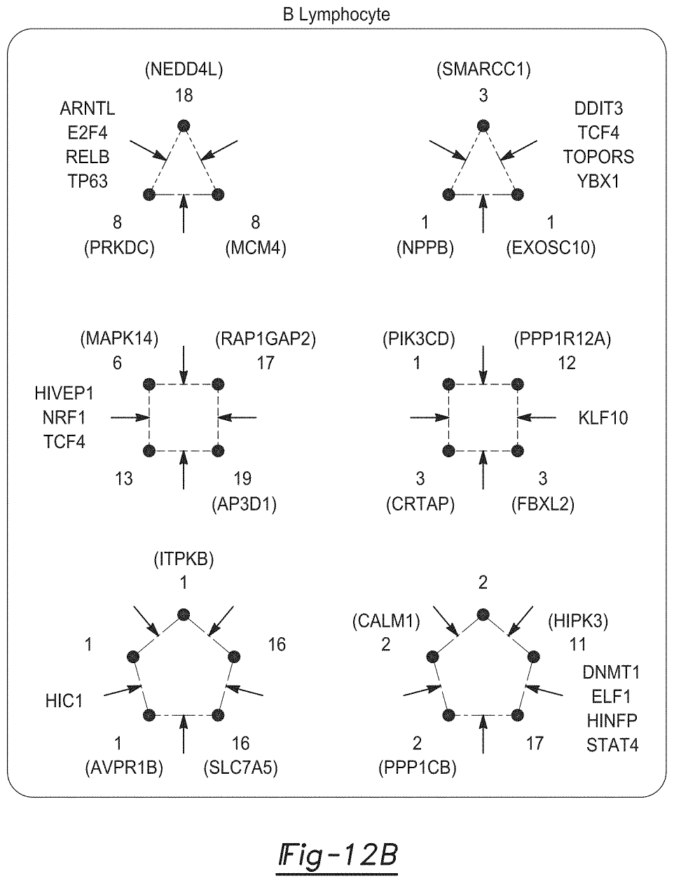

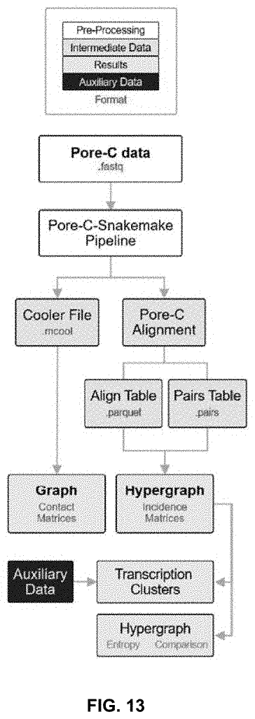

Continuing with the example described above in relation to FIGS. 5 and 6, 16,080 and 16,527 potential transcription clusters were identified from fibroblasts and B lymphocytes, respectively, using this technique. The majority of these clusters involved at least one expressed gene (72.2% in fibroblasts, 90.5% in B lymphocytes) and many involved at least two expressed genes (31.2% in fibroblasts, 58.7% in B lymphocytes). While investigating the colocalization of expressed genes in transcription clusters, it was found that over 30% of clusters containing multiple expressed genes had common transcription factors based on binding motifs (31.0% in fibroblasts, 33.1% in B lymphocytes) and that over half of these common transcription factors were master regulators (56.6% in fibroblasts, 74.7% in B lymphocytes). Two example transcription clusters derived from 3-way, 4-way, and 5-way contacts from both fibroblasts and B lymphocytes are shown in FIGS. 12A and 12B, respectively. These example clusters contain at least two genes which have at least one common transcription factor.

The criteria for potential transcription clusters was tested for statistical significance. That is, a test was conducted to determine whether the identified transcription clusters are more likely to include genes, and if these genes more likely to share common transcription factors, than arbitrary multi-way contacts in both fibroblasts and B lymphocytes. It was found that the transcription clusters were significantly more likely to include 1 gene and 2 genes than random multi-way contacts (p<0.01). In addition, transcription clusters containing 2 genes were significantly more likely to have common transcription factors and common master regulators (p<0.01). After testing all order multi-way transcription clusters, the 3-way, 4-way, 5-way, and 6-way (or more) cases were tested individually. It was found that all cases were statistically significant (p<0.01) except for clusters for common transcription factors or master regulators in the 6-way (or more) case for both fibroblasts and B lymphocytes. One can hypothesize that these cases were not statistically significant due to the fact that the large number of loci involved in these multi-way contacts will naturally lead to an increase of overlap with genes. This increases the likelihood that at least two genes will have common transcription factors or master regulators. Approximately half of transcription clusters with at least two genes with common transcription factors also contained at least one enhancer locus (˜51% and ˜44% in fibroblasts and B lymphocytes, respectively). This offers even further support that these multi-way contacts represented real transcription clusters.

Through advancements in sequencing technology, multi-way contacts within the genome can be captured and reported. Multiway contacts will become increasingly important within biological studies, as the relationship higher-order chromatin structures and genome function are intrinsically linked. Based on this information, medical diagnosis and treatment of patients can be made. Furthermore, this information can be used to reprogram cells of a patient, for example by introducing a given transcription factor into a particular cell of the patient.

The techniques described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for analyzing interactions in a human genome, comprising:
receiving a biological sample of a cell from a subject;
extracting read data from the biological sample, where the read data includes a set of reads; and
constructing, by a computer processor, a hypergraph from the read data, where each node in the hypergraph represents a locus and hyperedges in the hypergraph represent interactions between two or more loci.

2. The method of claim 1 wherein the read data has a length in range of 100 to 500 base pairs.

3. The method of claim 1 wherein the read data has a length selected from one of 100,000 base pairs, one million base pairs or 25 million base pairs.

4. The method of claim 1 further comprises
constructing an incidence matrix from the read data;
constructing a Laplacian matrix for the incidence matrix;
computing eigenvalues of the Laplacian matrix using eigendecomposition;
normalizing the eigenvalues of the Laplacian matrix; and
determining entropy of the hypergraph using the normalized eigenvalues.

5. The method of claim 4 wherein the eigenvalues of the Laplacian matrix are normalized such that Σλᵢ=1 and entropy is computed using Shannon entropy formula, where λᵢ is an eigenvalue of the Laplacian matrix.

6. The method of claim 1 further comprises
constructing an incidence matrix for the hypergraph;
for each multi-way contact in the incidence matrix, add a given multi-way contact to a set of potential transcription clusters in case where each locus associated with the given multi-way contact is accessible and at least one locus associated with the given multi-way contact is a binding site and the binding site is an indicator of transcription;
for each multi-way contact in the set of potential transcription clusters, add a particular multi-way contact to a set of transcription clusters in case where loci associated with the particular multi-way contact contains two or more expressed genes and have at least one common transcription factor; and
reporting multi-way contacts in the set of transcription clusters.

7. The method of claim 6 further comprises receiving chromatin accessibility data for the biological sample and determining whether locus are accessible from the chromatin accessibility data.

8. The method of claim 6 further comprises receiving binding data for the biological sample and determining whether a given locus is a binding site from the binding data, where the binding site is an indicator of transcription.

9. The method of claim 6 further comprises receiving gene expression data for the biological sample and determining whether a given loci contains two or more expressed genes from the gene expression data.

10. A method for analyzing interactions in a human genome, comprising:
receiving a first biological sample of a cell from a subject;
extracting read data from the first biological sample, where the read data includes a set of reads;
constructing a first hypergraph from the read data, where each node in the first hypergraph represents a locus and hyperedges in the first hypergraph represent interactions between two or more loci;
receiving a second biological sample of a cell from the subject;
extracting read data from the second biological sample, where the read data includes a set of reads;
constructing a second hypergraph from the read data, where each node in the second hypergraph represents a locus and hyperedges in the second hypergraph represent interactions between two or more loci; and
comparing the first hypergraph to the second hypergraph by computing a distance between the first hypergraph and the second hypergraph.

11. The method of claim 10 wherein the first biological sample is taken from a cell having a first cell type and the second biological sample is taken from a cell having a second cell type different from the first cell type.

12. The method of claim 10 wherein the first biological sample is taken from a cell having a given cell type at a given time and the second biological sample is taken from a cell of the subject having the same cell type but at a time different than the given time.

13. The method of claim 10 further comprises
constructing a first incidence matrix for the first hypergraph;
constructing a first normalized Laplacian matrix for the first incidence matrix;
computing a first set eigenvalues of the first normalized Laplacian matrix using eigendecomposition;
constructing a second incidence matrix for the second hypergraph;
constructing a second normalized Laplacian matrix for the second incidence matrix;
computing a second set of eigenvalues of the second normalized Laplacian matrix using eigendecomposition;
computing the distance between the first hypergraph and the second hypergraph using the first set of eigenvalues and the second set of eigenvalues.

14. The method of claim 13 wherein the first and the second normalized Laplacian matrix are constructed according to
$${\tilde{L}}_{i} = I - {D}_{i}^{- \frac{1}{2}} {H}_{i} {E}_{i}^{- 1} {H}_{i}^{T} {D}_{i}^{- \frac{1}{2}} ∈ {ℝ}^{n \times n}$$

15. A method for identifying transcription clusters in a human genome, comprising:
receiving a biological sample of a cell from a subject;
extracting read data from the biological sample, where the read data includes a set of reads; and
constructing a hypergraph from the read data, where each node in the hypergraph represents a locus and hyperedges in the hypergraph represent interactions between two or more loci;
constructing an incidence matrix for the hypergraph;
for each multi-way contact in the incidence matrix, add a given multi-way contact to a set of potential transcription clusters in case where each locus associated with the given multi-way contact is accessible and at least one locus associated with the given multi-way contact is a binding site and the binding site is an indicator of transcription;
for each multi-way contact in the set of potential transcription clusters, add a particular multi-way contact to a set of transcription clusters in case where loci associated with the particular multi-way contact contains two or more expressed genes and have at least one common transcription factor; and
reporting multi-way contacts in the set of transcription clusters.

16. The method of claim 15 further comprises receiving chromatin accessibility data for the biological sample and determining whether locus are accessible from the chromatin accessibility data.

17. The method of claim 15 further comprises receiving binding data for the biological sample and determining whether a given locus is a binding site from the binding data, where the binding site is an indicator of transcription.

18. The method of claim 15 further comprises receiving gene expression data for the biological sample and determining whether a given loci contains two or more expressed genes from the gene expression data.
